(19) United States
(12) Patent Application Publication
(10) Pub. No.: US 2003/0004130 A1
(43) Pub. Date: Jan. 2, 2003

(54) Homogeneous pharmaceutical compositions containing zidovudine and lamivudine

(76) Inventors: Malcolm Ross, Tel-Aviv (IL); David Caron, Nordia (IL); Tamar Gordon-Sommer, Givat Shmuel (IL)

Correspondence Address: Eitan, Pearl, Latzer & Cohen-Zedek, Suite 210, One Crystal Park, 2011 Crystal Drive, Arlington, VA, 22202-3709, US

(21) Appl. No.: 09/863,930

(22) Filed: May 24, 2001

(51) Int. Cl. A61K 31/7072; A61K 31/655; A61K 9/20

(52) U.S. Cl. 514/50; 514/151; 424/465

(57) ABSTRACT

The invention relates to methods of treating disease based upon pharmaceutical compositions, and processes for the preparation thereof, comprising zidovudine, or a derivative thereof and lamivudine, or a derivative thereof. The pharmaceutical composition is made by a wet granulation process whereby a binder is added to the zidovudine, or a derivative thereof. and lamivudine, or a derivative thereof, during the wet granulation process.

### FIELD OF THE INVENTION

The invention relates to pharmaceutical compositions, and processes for the preparation thereof, comprising zidovudine, or a derivative thereof; lamivudine, or a derivative thereof, and a binder.

### BACKGROUND OF THE INVENTION

In the control of viral infections in humans and animals, the use of enzyme inhibitors as anti-viral agents is increasing in demand. Both lamivudine and zidovudine are such anti-viral agents, being inhibitors of reverse transcriptase, an enzyme which is found in the pathway of development and reproduction of certain viruses, in particular retroviruses. Among the retroviruses which infect humans and which give rise to disease is the acquired immune deficiency syndrome virus or HIV, and strains thereof. Thus both lamivudine and zidovudine are administered to humans having an AIDS virus infection with the purpose of controlling the reproductive development of the virus and thereby treating the patient. The synergistic effect of these anti-viral agents has been described in WO96/30025 and it has been shown that it is difficult to obtain the required homogeneous dispersion of the active principles and requires special precautions as described in WO99/55372 and WO 98/18477.

The foregoing is particularly true of those medical conditions that require the use of several different drugs in the treatment regimen, which are difficult for the patient to ingest in the desired quantity. Thus, in designing a pharmaceutical formulation, an important consideration is the incorporation of a pharmacologically effective amount from each of a number of different medications within a single unit dose.

Another, related consideration, in designing a high dose oral pharmaceutical formulation is to maintain the dosage size at a degree which is amenable to high dose or multiple administration on a daily, basis for a period of months or years. Thus, it is desirable to have an orally prescribed drug in the form of vehicle suitable for oral administration, such as a tablet, which is small enough to be administered effectively and yet comprises an effective dose of the drug or drugs comprised therein. Tablet size is particularly important in the treatment of AIDS cases, where the subject is required to ingest a high number of medications throughout the day and on a chronic basis, usually for many years if not decades. Due to this problem, and the fact that such patients often suffer from dysphagia, e.g. difficulty swallowing, many patients do not take all of the prescribed dose of a medication and are thus placed in jeopardy from under-treatment of their medical condition. Continuity of therapy is particularly crucial in the treatment of viral infections. When compliance is poor, as due to tablets or capsules which are difficult to swallow, the danger of inundation of the body with viral particles becomes substantially more manifest. Thus, maintaining tablet size when increasing the amount of drug within the tablet, and diminishing tablet size while maintaining the same amount of drug within the tablet, are objectives bearing paramount clinical significance for millions of people.

In the traditional tablet form, and currently the only form approved for marketing, the anti-viral agent combination of zidovudine and lamivudine is formulated as a single oral dosage form comprising 300 mg zidovudine and 150 mg lamivudine, dosages which present certain unique difficulties. The therapeutic dose required for these drugs is as high as 900 mg/day zidovudine and 450 mg/day lamivudine, requiring essentially two to three tablets per day to achieve the desired schedule of dosing. Since the tablets are relatively large, swallowing the tablet becomes a once to thrice daily ordeal, particularly for the young or incapacitated patient, and in some cases may be impossible.

In the manufacture of any solid dosage form, the dispersion of the active substance or substances throughout the bulk powder is essential in order to ensure final content uniformity,

Thus for both good manufacturing practice, as well as regulatory authority guidelines [see Requirements for Uniformity of Final Blend—FDA Guidelines], require that an oral dosage form of a drug or drug combination be in the form of a homogeneous dispersion with respect to the active substances. In certain instances this can become problematic and it has been reported WO96/30025 that a preparation of zidovudine and lamivudine ordinarily requires the addition of a glidant in the course of the manufacture process in order to ensure the homogeneity of the final product. However, adding such excipients, such as glidants, invariably results in a tablet or capsule size of increased size and weight due to incorporation into the final product of excipients, such as glidants, added during the manufacture process. Thus, a recurring problem in the art of dispensary is to ensure that throughout the course of the manufacturing process the components are adequately mixed to provide a homogenous product, while at the same time preventing segregation of each of the components therein, thereby ensuring the correct amount of active material in each unit dosage form, while maintaining a small unit dosage form. The reliance on glidants to solve homogeneity issues is known but has been shown to be of variable value, depending very much on the particle size and shape of the materials being blended. Even after a homogeneous blend may be obtained, there are many instances of segregation occurring at a later stage of the process. This is particularly problematic where there are two or more active principles involved.

### SUMMARY OF THE INVENTION

This invention provides in one embodiment a homogeneous non-segregated composition comprising an amount of zidovudine, or a derivative thereof; an amount of lamivudine, or a derivative thereof; and an amount of a binder. In one embodiment the composition is a granulate.

This, invention provides in one embodiment a homogeneous non-segregated composition comprising zidovudine, or a derivative thereof, and lamivudine, or a derivative thereof, whereby said zidovudine, or a derivative thereof and lamivudine, or a derivative thereof, are directly combined in a single-step wet granulation process in the presence of a binder.

This invention provides in one embodiment a pharmaceutical composition, comprising a homogeneous non-segregated composition comprising an amount of zidovudine, or a derivative thereof; an amount of lamivudine, or a derivative thereof; and an amount of a binder.

This invention provides in one embodiment a pharmaceutical composition, comprising an homogeneous non-segregated composition comprising zidovudine, or a derivative thereof, and lamivudine, or a derivative thereof, whereby said zidovudine, or a derivative thereof, and lamivudine, or a derivative thereof, are directly combined in a single-step wet granulation process in the presence of a binder.

This invention provides in one embodiment a process for producing a granulate comprising an homogeneous composition comprising zidovudine, or a derivative thereof, and lamivudine, or a derivative thereof, whereby said zidovudine, or a derivative thereof, and lamivudine, or a derivative thereof, are directly combined in a-wet granulation process in the presence of a binder whereby the binding process effectively prevents segregation of said zidovudine, or a derivative thereof, and lamivudine, or a derivative thereof, in said composition.

This invention provides a pharmaceutical composition comprising said granulate comprising zidovudine, or a derivative thereof, and lamivudine, or a derivative thereof, whereby said zidovudine, or a derivative thereof, and lamivudine, or a derivative thereof, are directly combined in a wet granulation process in the presence of a binder whereby said binding process prevents segregation of said zidovudine, or a derivative thereof, and lamivudine, or a derivative thereof, in said composition.

### DETAILED DESCRIPTION OF THE INVENTION

The invention disclosed herein provides a highly concentrated, yet physically and chemically stable, composition of two or more anti-viral agents, namely zidovudine, or a derivative thereof, and lamivudine, or a derivative thereof, and a binder. This invention provides in one embodiment a process for producing a granulate comprising an homogeneous composition comprising zidovudine, or a derivative thereof, and lamivudine, or a derivative thereof, whereby said zidovudine, or a derivative thereof, and lamivudine, or a derivative thereof, are directly combined in a wet granulation process in the presence of a binder, whereby said process effectively prevents segregation of said zidovudine, or a derivative thereof, and lamivudine, or a derivative thereof, in said composition. The properties of the resulting pharmaceutical composition comprising the granulate are based on the ability to produce such granulate in the presence of a binder without any additional excipients.

Further, this invention provides in one embodiment a process for producing a granulate comprising an homogeneous composition comprising zidovudine, or a derivative thereof, lamivudine, or a derivative thereof, and abicavir, or a derivative thereof, whereby said zidovudine, or a derivative thereof, lamivudine, or a derivative thereof, and abicavir, or a derivative thereof, are directly combined in a wet granulation process in the presence of a binder whereby said process effectively prevents segregation of said zidovudine, or a derivative thereof, lamivudine, or a derivative thereof, and abicavir, or a derivative thereof in said composition.

The invention disclosed herein provides a process for the incorporation of zidovudine, or a derivative thereof and lamivudine, or a derivative thereof into a highly concentrated and homogeneous composition such that the granulate consists of 90% or more of the active ingredients. The composition in one embodiment may be in a pharmaceutical vehicle suitable for enteral administration including, but not only, granules, powders, tablets, capsules, and lozenges. Thus, in one embodiment, the pharmaceutical vehicle of the invention takes the form of a highly concentrated tablet or capsule which enables the delivery of high doses of zidovudine, or a derivative thereof, and lamivudine, or a derivative thereof, and a binder without adversely increasing the size of the oral tablet or capsule, thus providing an oral dosage form which contains a greater amount of drug but is not more difficult to handle or swallow.

The invention disclosed herein provides a process for the incorporation of zidovudine, or a derivative thereof, lamivudine, or a derivative thereof, and abicavir, or a derivative thereof, into a highly concentrated and homogeneous composition such that the granulate consists of 90% or more of the active ingredients. The composition in one embodiment may be in a pharmaceutical vehicle suitable for enteral administration including, but not only, granules, powders, tablets, capsules, and lozenges. Thus, in one embodiment, the pharmaceutical vehicle of the invention takes the form of a highly concentrated tablet or capsule which enables the delivery of high doses of zidovudine, or a derivative thereof, lamivudine, or a derivative thereof, and abicavir, or a derivative thereof, and a binder without adversely increasing the size of the oral tablet or capsule, thus providing an oral dosage form which contains a greater amount of drug but is not more difficult to handle or swallow.

Disclosed herein is a highly compact oral dosage form, and a process for the preparation thereof, comprising a biologically effective amount of two or more anti-viral agents. In one example, the anti-viral agents are lamivudine and zidovudine. In another example, the anti-viral agents are lamivudine, zidovudine and abacavir. In order to obtain such an oral dosage form, the invention provides a process for the combination of zidovudine, or a pharmaceutically acceptable derivative thereof, lamivudine, or a pharmaceutically acceptable derivative thereof, and abicavir, or a pharmaceutically acceptable derivative thereof, comprising a wet granulation step which ensures homogeneous dispersion while avoiding the use of bulk additives such as glidants to prevent segregation. In order to obtain such an oral dosage form, the invention provides a process for the combination of zidovudine, or a pharmaceutically acceptable derivative thereof, and lamivudine, or a pharmaceutically acceptable derivative thereof, comprising a wet granulation step which ensures homogeneous dispersion while avoiding the use of bulk additives such as glidants to prevent segregation. The invention, however, is not limited to the manufacture of zidovudine and lamivudine oral preparations, and is applicable to any anti-viral agent, i.e. abacavir, that is desirable for incorporating in high concentration into a composition or a pharmaceutically acceptable granule, in combination with zidovudine, or a derivative thereof, and lamivudine, or a derivative thereof, and which composition, granule, or oral dosage form has the characteristics of high concentration of the zidovudine, or a derivative thereof; lamivudine, or a derivative thereof (e.g. greater than 90% by dry weight) and homogeneity with respect to the pharmacologically active agents.

For purposes of the present invention, a wet granulation process is exemplified in [Bandelin in Compressed tablets by wet granulation p149-Pharmaceutical Dosage Forms: Tablets Vol 1 2^{nd} ed. Marcel Dekker, 1989].

It is well known to those skilled in the art that wet granulation is a preferred procedure to ensure content uniformity in the manufacture of low dose products. It has also been demonstrated that problems of desegregation can occur in high dose products and that such problems must be controlled by the addition of additional excipients such as glidants. Surprisingly, it has been found that a homogenous and stable product can be prepared from a combination of zidovudine, or a derivative thereof and lamivudine, or a derivative thereof, by a wet granulation process such that the resulting granulate does not undergo desegregation during further processing, even in the absence of glidants, by the use of a one or more pharmaceutically acceptable binders. Preferably, the binder is dissolved in the appropriate solvent prior to, or in the course of, adding either or both of the anti-viral drugs. In another embodiment, the binder is mixed with either one or both of the anti-viral drugs prior to addition of the solvent. For granulating, a suitable blender, high shear mixer, or spray process in a fluid bed granulator is suitable. The granulate is then dried, mixed with additional excipients as desired, and either filled into capsules or compressed. Once the granulate has been formed there is no further possibility of segregation and consequently further processing will not affect the ratio of the active substances and no segregation between them can occur. In the case of the zidovudine, or a pharmaceutically acceptable derivative thereof, and lamivudine, or a pharmaceutically acceptable derivative thereof, such a process affords a granulate product with good quality attributes and that can be compressed to produce a tablet of required hardness and friability, while uniform dispersion of the active components is maintained and the desired high concentration per weight of the active components is achieved, thereby providing the miniaturized, pharmaceutically acceptable oral dosage configuration of the invention comprising an effective amount of both zidovudine, or a pharmaceutically acceptable derivative thereof, and lamivudine, or a pharmaceutically acceptable derivative thereof.

Thus, whereas formulations using a glidant to prevent segregation require the addition of considerable amounts of excipients in order to obtain a tablet of suitable properties, the use of the granulated zidovudine/lamivudine combination allows the manufacture of significantly smaller tablets. For example, the commercially known product, as disclosed in patent WO 98/18477, contains 300 mg of zidovudine and 150 mg lamivudine in a total tablet weight of 750 mg so that the excipients account for over half of the tablet weight. By contrast, the tablet provided by the invention disclosed herein contains a concentration of at least 80%, and as high as 97% [w/w] of active principles. Since, as provided by the present invention, a reduction in the amount of tablet excipients is accompanied by a concomitant reduction in tablet size, the result being that the tablet is more easily swallowed, thus increasing patient comfort and compliance. This is of particular importance in the case of drugs used in the treatment of HIV infection as patients may exhibit odynophagia [pain with swallowing] or dysphagia [difficulty swallowing].

Zidovudine is described in J. P. Horowitz et al J. Org. Chem. 29 [1964] p. 20176 and its use in the treatment of HIV infections has been well documented [Physicians Desk Reference]. The chemical name for the compound is 3′-Azido-3′-deoxythymidine or Azydothymidine. Zidovudine is marketed under the trade name RETROVIR® as a treatment for retroviral infections.

Lamivudine is a closely related compound to Zidovudine and is used for the same purposes. Lamivudine is described in patents GR 3608606 and U.S. Pat. No. 4,724,232, incorporated herein by reference. Synthesis of lamivudine is described in U.S. Pat. No. 5,047,407, incorporated herein by reference. The chemical name for the compound is (2R-cis)-4Amino-1-[2-(hydroxymethyl)-1,3-oxathiolan-5-yl]-2(1H)-pyrimidone. Lamivudine is marketed under the trade name EPIVIR® as a treatment for retroviral infections.

A combination of zidovudine and lamivudine is marketed by GLAXO Smith Klein Beecham as COMBIVIR®.

The invention disclosed herein provides a composition for use in producing a pharmaceutical vehicle comprising zidovudine, or a pharmaceutically acceptable derivative thereof, and lamivudine, or a pharmaceutically acceptable derivative thereof, in a fixed ratio, thereby allowing for the formation of more compact dosage forms. Thus, as described herein, a substantially more concentrated, pharmaceutically acceptable tablet form of the drug may be produced. The property of increased concentration, according to the present invention, is achieved while preserving the pharmaceutically acceptable property of homogeneity of the active components of the dosage form. According to the present invention, such a dosage form is chemically stable and, when in tablet form, is durable upon storage and handling.

In one embodiment, the invention provides a pharmaceutically acceptable composition comprising, in a high concentration, zidovudine, or a pharmaceutically acceptable derivative thereof, and lamivudine, or a pharmaceutically acceptable derivative thereof, for treating a viral infection in a subject in need of administration thereof, and which pharmaceutically acceptable composition can be delivered from a compact dosage form manufactured to a high degree of physical and chemical stability and homogeneity, thus ensuring reliable dosing of the drug without compromising patient compliance. The composition of the invention may be used to manufacture either high dose tablets or capsules of an anti-viral agent combination without increasing the tablet or capsule size, or substantially smaller tablets or capsules containing the regular unit dose of a combination of zidovudine, or a derivative thereof, and lamivudine, or a derivative thereof.

In another embodiment, the present invention provides a dosage form adapted for oral administration of a combination of zidovudine, or a pharmaceutically acceptable derivative thereof, lamivudine, or a pharmaceutically acceptable derivative thereof, which dosage form is compact, and which pharmaceutically acceptable dosage form can be manufactured to a high degree of physical and chemical stability, and homogeneity, thus ensuring reliable dosing of the drug.

In another embodiment, the present invention provides a method for treating a viral infection by orally administering a pharmaceutically acceptable composition of a combination of zidovudine, or a derivative thereof, and lamivudine, or a derivative thereof, in a compact dosage. form, thereby reducing patient discomfort, enhancing patient compliance, improving the reliability of dosing, or a combination thereof.

In another embodiment, the present invention provides a process for the manufacture of an oral solid dosage form of a combination of zidovudine, or a pharmaceutically acceptable derivative thereof, and lamivudine, or a pharmaceutically acceptable derivative thereof, which is economical to manufacture and reliably reproducible.

In another embodiment, the present invention provides an oral solid dosage form of a combination of zidovudine, or a pharmaceutically acceptable derivative thereof, and lamivudine, or a pharmaceutically acceptable derivative thereof, in a compact dosage form which maintains its integrity during storage, while possessing suitable disintegration and dissolution properties, e.g., to gastrointestinal fluid, when administered in therapeutic doses.

In another embodiment, the present invention provides an improved oral dosage form of a combination of zidovudine, or a pharmaceutically acceptable derivative thereof, and lamivudine, or a pharmaceutically acceptable derivative thereof, in a compact dosage form which is physically and chemically stable and which has hardness and friability characteristics suitable for use in commercially available preparations.

In another embodiment, the present invention provides a solid dosage form which includes two or more active ingredients for the treatment of a subject afflicted with one or more viral infections.

In another embodiment, the present invention provides an improved oral dosage form of a combination of zidovudine, or a pharmaceutically acceptable derivative thereof, and lamivudine, or a pharmaceutically acceptable derivative thereof, in a compact dosage form, which is more readily administered, e.g., easier to handle and to swallow, thus allowing for safer dosing of the active agent and therefore an improved regimen of therapy by oral administration of zidovudine, or a derivative thereof and lamivudine, or a derivative thereof.

According to the invention, the use of a wet granulation process in combination with a suitable binder for the manufacture of a composition comprising a combination of zidovudine, or a derivative thereof, and lamivudine, or a derivative thereof, as well as for granules and.pharmaceutical vehicles comprising a combination of zidovudine, or a derivative thereof, and (lamivudine, or a derivative thereof, prevents segregation of the zidovudine, or a derivative thereof, and lamivudine, or a derivative thereof, from among the other anti-viral ingredients of the composition. Thus, the use of the wet granulation process for combining the zidovudine, or a derivative thereof, and lamivudine, or a derivative, thereof allows for the homogeneous dispersion of the agents in the resultant pharmaceutically acceptable composition.

In one embodiment of the process according to the invention, the zidovudine, or a derivative thereof, and lamivudine, or a derivative, and the desired amount of excipients, including at least one binder, is added in a suitable mixer, such as a high shear mixer, a blender, or an apparatus for a spray process of granulation. The granulate is then dried, milled and mixed with additional excipients as desired and either filled into capsules or compressed. To achieve a small solid dosage form, the amount of added excipients is held to a minimum. The product of such a process affords a granulation with good quality attributes that can be compressed to afford a tablet of required small size, hardness and friability. Suitable materials for a manufacturing a combination of zidovudine, or a pharmaceutically acceptable derivative thereof, and lamivudine, or a pharmaceutically acceptable derivative thereof, in a compact dosage form, according to the present invention are pharmaceutically acceptable excipients including, but not only, the following: binders, such as cellulose and its derivatives, e.g. ethyl cellulose hydroxypropylmethyl cellulose, hydroxypropylcellulose, hydroxyethyl cellulose, sodium carboxymethyl cellulose, starches or derivatives, polyvinyl pyrrolidone, natural gums, corn syrup, polysaccharides (including acacia, tragacanth, guar, and alginates), gelatin, or a combination thereof; lubricants, such as magnesium stearate, calcium stearate, aluminum stearate, stearic acid, calcium oleate, talc, mineral oil, waxes, glyceryl behenate, potassium stearyl fumarate, sodium stearyl fumarate, hydrogenated vegetable oils, or a combination thereof; and diluents, such as lactose, cellulose, starch, calcium phosphate, or a combination thereof Said lubricants are commonly included in the final product in amounts of less than 1% by weight.

The term “a granulate” is intended to mean the granulate obtainable by using the wet granulation method and has the general meaning as disclosed in e.g. Remington: The Science and Practice of Pharmacy, 19^{th} Edition (1995) and/or in Handbook of Pharmaceutical Granulation Technology, Chapter 7, “Drugs and the Pharmaceutical Sciences”, vol. 81, 1997. The term “a granule” refers herein to the one or more particulate structural components comprising a granulate, in either a dry state, a semi-dry state, a wet state, or a combination thereof. A granule or granules may have any suitable size, depending on the carriers and/or equipment used and the preparation of granules with a particular size and structure is within the technical knowledge of the skilled person.

The term “a wet granulation method” represents a conventional way of making a granule, granules, or a granulate and is disclosed in e.g. Remington: The Science and Practice of Pharmacy, 19^{th} Edition (1995) and/or in Handbook of Pharmaceutical Granulation Technology, Chapter 7, “Drugs and the Pharmaceutical Sciences”, vol. 81, 1997. The wet method usually comprises the steps of weighing, mixing, granulation, screening the damp mass, and drying. The wet method may further comprise an additional step or steps of dry screening, lubrication, compression, or a combination thereof.

The terms “granulation” and “granulation of the mixture” are intended to have the usual meaning, as disclosed in Remington: The Science and Practice of Pharmacy, 19^{th} Edition (1995) and/or in Handbook of Pharmaceutical Granulation Technology, Chapter 7, “Drugs and the Pharmaceutical Sciences”, vol. 81, 1997; and include one or more steps of dry blending and wet massing performed prior to or after a granulation step.

The term “drying the mixture” is intended to have its usual meaning, as disclosed in Remington: The Science and Practice of Pharmacy, 19^{th} Edition (1995) and/or in Handbook of Pharmaceutical Granulation Technology, Chapter 7, “Drugs and the Pharmaceutical Sciences”, vol. 81, 1997; and comprises drying the granulation mixture in a conventional manner either inside or outside mixing means, such as but not only a granulator or a high shear mixer, by placing the moist granulation mixture, such as but not limited to, drying cabinets with circulating air current and thermostatic heat control.

The term “processing the granulate” is intended to mean the further conventional processing of the granulate into an oral solid dosage formulation as disclosed in Remington: The Science and Practice of Pharmacy, 19^{th} Edition (1995) and/or in Handbook of Pharmaceutical Granulation Technology, Chapter 7, “Drugs and the Pharmaceutical Sciences”, vol. 81, 1997; and comprises, but is not limited to, reducing the granules or granulate to a particular a particular size, lubrication, and compressing into tablets, lozenges, powders, or filling into gelatin capsules.

The terms “pharmaceutically acceptable” and “or a salt thereof” represent forms of an ingredient that are physiologically acceptable for pharmaceutical use, including pharmaceutically acceptable salts thereof.

The terms “an oral solid dosage formulation” or “a pharmaceutically acceptable vehicle suitable for enteral administration” are intended to mean such solid dosage formulations as disclosed in Remington: The Science and Practice of Pharmacy, 19^{th} Edition (1995) and/or in Handbook of Pharmaceutical Granulation Technology, Chapter 7, “Drugs and the Pharmaceutical Sciences”, vol. 81, 1997; and comprises, but is not limited to, tablets, including comestibles, capsules, pills, lozenges, troches, cachets, and pellets.

According to another embodiment of process of the invention, the combination of zidovudine, or a pharmaceutically acceptable derivative thereof, and lamivudine, or a pharmaceutically acceptable derivative thereof, are mixed together and the granulating fluid may be added, for example, by drops or spraying. The resulting mixture is then dried to form a stable composition of the zidovudine, or a derivative thereof, lamivudine, or a derivative thereof homogeneously combined with a binder excipient. Suitably dried, the composition consists of stable, homogeneous granules suitable for further incorporation into a solid dosage form.

According to another embodiment of the process of the invention, the zidovudine, or a pharmaceutically acceptable derivative thereof, and lamivudine, or a pharmaceutically acceptable derivative thereof, and the solid binder are combined prior to addition of the granulating fluid which may, in this case, be water.

As used herein, the term “granulating fluid” refers to any fluid composition which is capable of causing adhesion between zidovudine and lamivudine. In a preferred embodiment of the present pharmaceutical composition, especially wherein a granulate comprising the composition is compressed into tablet form, suitable ingredients are: zidovudine, lamivudine, crospovidone, and, as granulating fluid, water.

In the foregoing embodiments, the steps of the process may be performed in an order which allows one skilled in the art to obtain the composition, granules thereof, and pharmaceutically acceptable vehicles thereof, in expeditious manner which does not diminish from the desired properties of the final product. Thus, for example, the drugs and the excipient may first be dry mixed, together with any additional excipients, as required. The mixed powders are then granulated by wetting with a granulating fluid. In another embodiment, the drug or excipients are first combined with the granulating fluid and then further excipient and/or drug is added to form a blend. Thus the methods of the invention for preparing stable, homogeneous anti-viral compositions, granules, and tablets thereof, comprise steps that may be performed in a variety of sequences in order to achieve the desired product. Determining the order of the steps for implementing the process of the invention may be readily appreciated by one skilled in the art of dispensary.

Similarly, the agent or agents may be added in dry form to the other ingredients, or first dissolved in the granulating fluid prior to addition of the other ingredients to form a blend. The addition of further excipients, including binders, lubricants, carriers, disintegrants, diluents, anti-adhesives, or a combination thereof, may be similarly performed.

According to preferred embodiments of the invention, the granulating fluid is comprised of one or more solvents, preferably water. A tableting composition is preferably produced from the granulated mass or granules obtained directly from the dried granulate, or from a granulate, by subjoinment with a tableting binder such as sodium carboxymethyl cellulose, a lubricant such as magnesium stearate, or disintegrants such as croscarmellose sodium. There are many other pharmaceutically acceptable tableting binders, lubricants and disintegrants well known in the pharmaceutical arts which are usable in the production of the tablets of the present invention.

Such tableting composition is then fed to a multi-station tablet press for compression into tablets. The tablets of the invention show satisfactory hardness and friability and they comply with the usual standards with respect to disintegration time

According to the present disclosure, the invention provides stable, homogeneous granules comprising the composition according to any one of the foregoing embodiments.

According to the present disclosure, the invention provides a pharmaceutical vehicle suitable for enteral administration comprising a composition according to any one of the foregoing embodiments of the invention, wherein said vehicle is selected from the group consisting of tablets, capsules, lozenges, powders, or granules.

In one embodiment, the composition of the invention comprises zidovudine, or a derivative thereof, lamivudine, or a derivative thereof, one or more pharmaceutically acceptable binders, one or more pharmaceutically acceptable carriers, and optionally other therapeutic agents. Formulations of the zidovudine, or a derivative thereof; lamivudine, or a derivative thereof described herein encompass those directed to administration by various oral routes, including a sustained release formulation thereof. Preferably, for the purpose of implementing the methods of the invention, an oral dosage form is administered. The carrier(s) must be “acceptable” in that it has to be compatible with, and stabilizing of, the active ingredient(s) of the formulation, and not deleterious to the subject to be treated (“pharmaceutically acceptable”).

In general, the formulations for tablets or powders may be prepared by uniformly and intimately blending the active ingredient with finely divided solid carriers, and then, if necessary, as in the case of tablets, forming the product into the desired shape and size.

The present pharmaceutical composition, together with processes for its preparation, will now be described by way of example only.

### EXAMPLES

Tablets prepared in accordance with these Examples exhibited pharmaceutically acceptable properties with regard to stability, disintegration times and dissolution rates.

### Example 1

Tablets were compressed from the following ingredients based on an aqueous wet granulation process:

TABLE 1

| Material Name | mg/tab |
|---|---|
| Lamivudine | 150 |
| Zidovudine | 300 |
| Polyvinylpyrrolidone | 22.5 |
| Croscarmellose sodium | 81.9 |
| Magnesium Stearate | 5.6 |
| Total Weight of Core Tablet | 560 |

The corresponding quantities of the materials in Table 1 are weighed from bulk containers. Lamivudine and zidovudine are granulated with a solution of binder (e.g. polyvinylpyrrolidone), dried, and then milled through a 20 mesh sieve. The milled granulate is then mixed with a disintegrant (e.g. croscarmellose sodium) in a blender (e.g. V-shaped) for about 5 minutes. Magnesium stearate, preliminarily sieved through a 30 mesh sieve is added to the mixture and blending is continued for about 2 minutes. The lubricated blend is then compressed using a suitable rotary tablet press typically using a Manesty Betapress or equivalent. In process controls for tablet weight and hardness are applied at appropriate intervals throughout the compression run and adjustments to the tablet press are made as necessary. The uncoated tablets had a friability of less than 0.8% and a crushing strength of at least 10 kilopond. The tablet core volume is 423 mm³, compared to 640 mm³ for the brand leader.

### Example 2

The formula of Example 1 was followed except that the Polyvinylpyrrolidone was added together with the Lamivudine and Zidovudine and the powder mixture immediately granulated through an aqueous granulation procedure. The granulate is dried and milled through a 20 mesh sieve. The milled granulate is then mixed with a disintegrant (e.g. croscarmellose sodium) in a blender (e.g. V-shaped) for about 5 minutes. Magnesium stearate, preliminarily sieved through a 30 mesh sieve is added to the mixture and blending is continued for about 2 minutes. The lubricated blend is then compressed using a suitable rotary tablet press typically using a Manesty Betapress or equivalent. In process controls for tablet weight and hardness are applied at appropriate intervals throughout the compression run and adjustments to the tablet press are made as necessary, The uncoated tablets had a friability of less than 0.8% and a crushing strength of at least 10 kilopond. The tablet core volume is 423 mm³, compared to 640 mm³ for the brand leader.

TABLE 2

| Material Name | mg/tab |
|---|---|
| Lamivudine | 150 |
| Zidovudine | 300 |
| Polyvinylpyrrolidone | 22.5 |
| Croscarmellose sodium | 81.9 |
| Magnesium Stearate | 5.6 |
| Total Weight of Core Tablet | 560 |

### Example 3

The procedure of Example 1 was followed except that the following composition was used, in which tablet weight has been reduced by adding crospovidone instead of croscarmellose:

TABLE 3

| Material Name | mg/tab |
|---|---|
| Lamivudine | 150 |
| Zidovudine | 300 |
| Polyvinylpyrrolidone | 22.5 |
| Crospovidone | 25.25 |
| Magnesium Stearate | 5.6 |
| Total Weight of Core Tablet | 505 |

In this formulation a core tablet weight of 505 mg is compressed to yield a tablet containing 89% by weight of zidovudine, or a derivative thereof; lamivudine, or a derivative thereof. The tablet core volume is 423 mm³, compared to 640 mm³ for the brand leader, The uncoated tablets had a friability of less than 0.8% and a crushing strength of at least 10 kilopond.

### Example 4

The procedure was based on Example 1 and the following composition was used:

| Material Name | mg/tab |
|---|---|
| Lamivudine | 150 |
| Zidovudine | 300 |
| Hydroxypropyl cellulose | 22.5 |
| Croscarmellose sodium | 81.9 |
| Magnesium Stearate | 5.6 |
| Total Weight of Core Tablet | 560 |

The corresponding quantities of the materials in Table 4 are weighed from bulk containers. Lamivudine and Zidovudine are granulated with a solution of binder (e.g. Hydroxypropylcellulose), dried, and then milled through a 20 mesh sieve. The milled granulate is then mixed with a disintegrant (e.g. croscarmellose sodium) in a blender (e.g. V-shaped) for about 5 minutes. Magnesium stearate, preliminarily sieved through a 30 mesh sieve is added to the mixture and blending is continued for about 2 minutes, The lubricated blend is then compressed using a suitable rotary tablet press typically using a Manesty Betapress or equivalent. In process controls for tablet weight and hardness are applied at appropriate intervals throughout the compression run is and adjustments to the tablet press are made as necessary. The uncoated tablets had a friability of less than 0.8% and a crushing strength of at least 10 kilopond. The tablet core volume is 423 mm³, compared to 640 mm³ for the brand leader.

What we claim is:

1. An homogeneous non-segregated composition comprising an amount of zidovudine, or a derivative thereof; an amount of lamivudine, or a derivative thereof; and an amount of a binder.

2. The homogeneous non-segregated composition of claim 1, wherein the composition is a granulate.

3. The homogeneous non-segregated composition of claim 1, wherein said binder is povidone, polyvinyl pyrrolidone, starch, starch derivatives, acacia, tragacanth, gelatin, cellulose derivatives, or a mixture thereof.

4. The homogeneous non-segregated composition of claim 1, wherein the amount of the binder of said composition ranges from 0.5% to 25% by dry weight of said composition.

5. The homogeneous non-segregated composition of claim 1, wherein said zidovudine, or a derivative thereof and amount of lamivudine, or a derivative thereof comprise at least 60% by dry weight of said composition and said binder comprises from 0.5% to 5% by dry weight of said composition.

6. The homogeneous non-segregated composition of claim 1, wherein said zidovudine, or a derivative thereof and amount of lamivudine, or a derivative thereof comprises at least 60% by dry weight of said composition and said binder comprises from 5% to 10% by dry weight of said composition.

7. The homogeneous non-segregated composition of claim 1, wherein said zidovudine, or a derivative thereof and amount of lamivudine, or a derivative thereof comprises at least 60% by dry weight of said composition and said binder comprises from 10% to 25% by dry weight of said composition.

8. An homogeneous non-segregated composition comprising zidovudine, or a derivative thereof; and lamivudine, or a derivative thereof, whereby said zidovudine, or a derivative thereof and lamivudine, or a derivative thereof, are directly combined in a single-step wet granulation process in the presence of a binder.

9. The homogeneous non-segregated composition of claim 8, wherein said binder is povidone, polyvinyl pyrrolidone, starch, starch derivatives, acacia, tragacanth, gelatin, cellulose derivatives, or a mixture thereof.

10. The homogeneous non-segregated composition of claim 8, wherein the amount of the binder of said composition ranges from 0.5% to 25% by dry weight of said composition.

11. The homogeneous non-segregated composition of claim 8, wherein said zidovudine, or a derivative thereof and amount of lamivudine, or a derivative thereof comprise at least 60% by dry weight of said composition and said binder comprises from 0.5% to 5% by dry weight of said composition.

12. The homogeneous non-segregated composition of claim 8, wherein said zidovudine, or a derivative thereof and amount of lamivudine, or a derivative thereof comprises at least 60% by dry weight of said composition and said binder comprises from 5% to 10% by dry weight of said composition.

13. The homogeneous non-segregated composition of claim 8, wherein said zidovudine, or a derivative thereof and amount of lamivudine, or a derivative thereof comprises at least 60% by dry weight of said composition and said binder comprises from 10% to 25% by dry weight of said composition.

14. The homogeneous non-segregated composition of claim 8 further comprising a carrier or diluent.

15. The homogeneous non-segregated composition of claim 14, wherein said carrier is a sugar, a disaccharide, lactose, a polysaccharide, calcium phosphate, cellulose, a derivative of cellulose, lactose, or a combination thereof.

16. The homogeneous non-segregated composition of claim 8, further comprises a disintegrant, a lubricant, or a combination thereof.

17. The homogeneous non-segregated composition of claim 16, herein said disintegrant is sodium starch glycolate, pregelatinized starch, cross-linked povidone, croscarmelose sodium, calcium pectinate, or a combination thereof.

18. The homogeneous non-segregated composition of claim 8, wherein said composition is further coated with a pharmaceutically acceptable coating.

19. The homogeneous non-segregated composition of claim 18, wherein said coating is a sustained release coating which substantially prevents the release of said derivatives prior to arrival in the small intestine.

20. A pharmaceutical composition, comprising the composition of claim 1-19 and a pharmaceutical acceptable carrier or diluent.

21. A pharmaceutical vehicle suitable for enteral administration comprising the pharmaceutical composition according to claim 20, wherein said vehicle is selected from the group consisting of tablets, capsules, lozenges, powders, or granules.

22. The pharmaceutical composition of claim 20, comprising zidovudine, or a pharmaceutically acceptable derivative thereof, in an amount ranging from 45 to 60% by dry weight of said vehicle and lamivudine, or a pharmaceutically acceptable derivative thereof, in an amount ranging from 25 to 30% by dry weight of said vehicle, wherein the total core weight of said vehicle ranges from 200 to 700 mg.

23. The pharmaceutical composition of claim 20, comprising zidovudine, or a pharmaceutically acceptable derivative thereof, in an amount ranging from 100 mg to 400 mg and lamivudine, or a pharmaceutically acceptable derivative thereof, in an amount ranging from 50 mg to 200 mg, wherein the size of said vehicle ranges from 423 mm³ to 640 mm³.

24. A process for producing a granulate comprising a homogeneous composition comprising zidovudine, or a derivative thereof and lamivudine, or a derivative thereof, whereby said zidovudine, or a derivative thereof and lamivudine, or a derivative thereof are directly combined in a single-step wet granulation process in the presence of a binder whereby said binder effectively prevents segregation of said zidovudine, or a derivative thereof and lamivudine, or a derivative thereof in said composition.

25. The process according to claim 24, whereby the amount of said binder ranges from 0.5% to 25% by dry weight of said granulate.

26. The process according to claim 25, whereby the amount of said zidovudine, or a derivative thereof and lamivudine, or a derivative thereof is equal to at least 60% by dry weight of said granulate and said binder comprises from 0.5% to 5% by dry weight of said granulate.

27. The process according to claim 25, whereby the amount of said zidovudine, or a derivative thereof and lamivudine, or a derivative thereof is equal to at least 60% by dry weight of said granulate and the amount of said binder ranges from 5% to 10% by dry weight of said granulate.

28. The process according to claim 25, whereby the amount of said zidovudine, or a derivative thereof and lamivudine, or a derivative thereof equals at least 60% by dry weight of said granulate and said binder comprises from 10% to 25% by dry weight of said granulate.

29. The process according to claim 25, whereby said binder is povidone, polyvinyl pyrrolidone, starch, starch derivatives, acacia, tragacanth, gelatin, cellulose derivatives, or a mixture thereof.

30. The process according to claim 25, whereby said carrier is a sugar, a disaccharide, lactose, a polysaccharide, calcium phosphate, cellulose, a derivative of cellulose, lactose, or a combination thereof.

31. The process according to claim 25, whereby said process further comprises the step of adding a pharmaceutically acceptable disintegrant, a pharmaceutically acceptable lubricant, or a combination thereof.

32. The process according to claim 31, whereby said disintegrant is sodium starch glycolate, pregelatinized starch, cross-linked povidone, croscarmelose sodium, calcium pectinate, or a combination thereof.

33. The process according to claim 25, whereby said granulate is further coated with a pharmaceutically acceptable coating.

34. The process according to claim 31, whereby said coating is a sustained release coating which substantially prevents the release of said derivatives prior to arrival in the small intestine.

35. A pharmaceutical composition comprising said granulate of claim 25.
